(19) United States
(12) Patent Application Publication
(10) Pub. No.: US 2005/0090723 A1
(43) Pub. Date: Apr. 28, 2005

(54) Method and apparatus for non-invasive measuring of physiological glucose concentration in bodies of humans or animals

(76) Inventor: Nassar Saeed, Amman (JO)

Correspondence Address: John H. Oltman;Oltman, Flynn & Kubler, 915 Middle River Drive #415, Ft. Lauderdale, FL, 33304-3585, US

(21) Appl. No.: 10/690,967

(22) Filed: Oct. 23, 2003

(51) Int. Cl. A61B 5/00

(52) U.S. Cl. 600/316; 600/310

(57) ABSTRACT

Method and apparatus for non-invasive measurement of the blood glucose concentration (or other selected physiological content) of a body tissue specimen by irradiating the specimen with modulated laser light, and receiving light re-emitted from the specimen to produce an electrical signal which controls the duration of laser energization and is decoded before operating a counter from a variable frequency pulse generator.

### BACKGROUND OF THE INVENTION

1. Field of the Invention

This invention relates to non-invasive measurement of the physiological glucose concentration in a human or animal tissue specimen, such as a person's finger.

2. Prior Art

To determine the glucose content, particularly the blood glucose level, of human and animal tissues various methods invasive methods have been used which involve spectrophotometric or other analysis of the tissue. The invasive nature of such methods has been a principal drawback to their use.

Various non-invasive methods of determining the blood glucose level in humans have been proposed heretofore which are cumbersome, difficult to use, and require frequent re-calibration of instruments involved in the method.

Also, it has been proposed heretofore to direct high frequency radio waves onto the specimen and analyze the resulting energy emitted from the specimen to determine its glucose level. This radio frequency technique is complicated and requires complex circuitry.

### SUMMARY OF THE INVENTION

The present invention relates to a simple and reliable method and apparatus for non-invasive testing which is applicable to various types of human or animal tissue specimens, requires no calibration, and can be readily operated by a lay person who does not have special expertise.

One aspect of the present invention is a non-invasive method of measuring the glucose concentration in the body of a human being or an animal. In accordance with this aspect, a principal object of this invention is to provide a novel and advantageous method of measuring the physiological glucose concentration of a body tissue specimen, particularly an in vivo specimen, such as a person's finger.

Another aspect of this invention is an apparatus for conducting non-invasive testing of a body tissue specimen, particularly an in vivo specimen, for its glucose content. In accordance with this aspect, another principal object of this invention is to provide a novel and advantageous apparatus for measuring the glucose concentration of a body tissue specimen.

Further objects and advantages of this invention will be apparent from the following detailed description of a presently preferred embodiment thereof, with reference to the single Figure of the accompanying drawing.

### BRIEF DESCRIPTION OF THE DRAWING

FIG. 1 is a schematic circuit diagram of apparatus in accordance with the present invention for performing the method of this invention.

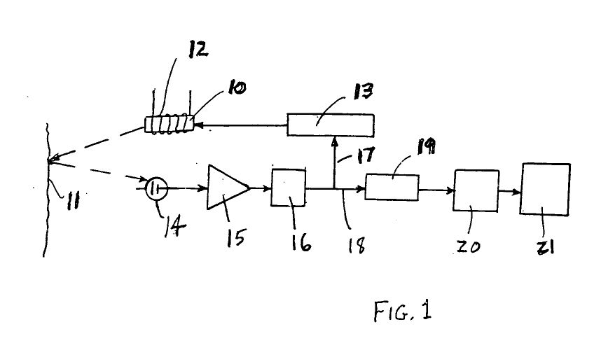

### DETAILED DESCRIPTION OF THE INVENTION

Before explaining the present invention in detail it is to be understood that the invention is not limited in its application to the particular arrangement shown and described since the invention is capable of other embodiments. Also, the terminology used herein is for the purpose of description and not of limitation.

The illustrated apparatus of this invention comprises a laser diode 10 of known design which transmits red light to irradiate a body tissue specimen 11, such as a human person's finger in vivo. The position of the laser beam's impingement on the specimen is under the control of a signal applied to a coil of fine wire 12 wound around the laser. The laser does not focus the laser light on a very specific point on the surface of the specimen, and the time period of the laser's energization is very short so that there is no danger of damaging the specimen tissue. The laser is energized by a driver 13, which preferably includes a unijunction transistor. The driver modulates the laser 10 at a suitable high frequency in the audio range, such as 10 MHz.

Laser light incident on the specimen 11 is selectively absorbed by the material of the specimen, depending upon the type and concentration of chemicals in the specimen. In the present invention glucose is the chemical of particular interest in the in vivo specimen. In response to its irradiation with laser light, the specimen emits red light which has an intensity that depends on the concentration of glucose in the specimen's blood. The light coming from the specimen impinges on a photocell 14 positioned close to the laser. This photocell converts the incident light coming from the specimen 11 into an electrical output signal which is amplified by an amplifier 15 and filtered by a filter 16. Preferably, amplifier 15 and filter 16 are embodied in an integrated circuit amplifier of known design. The amplified and filtered signal is applied via line 17 to the laser driver 13 to control the duration of the energization of the laser 10, thereby determining the length of time for the test of a particular specimen.

The amplified and filtered output signal of the photocell also is applied through line 18 to the input of a decoder 19, which operates to effectively extract from this signal the pertinent information about the body tissue specimen, in this case, its glucose content. The decoder, which preferably is an integrated circuit, is programmed to receive needed information from the light with specific density and frequency which is received by photocell 14 from the specimen and amplified and filtered as described, and converts this information into an output signal which is applied to a variable frequency pulse generator 20 to produce a string of pulses which are counted in a counter circuit 21 of known design. The pulse input to counter 21 ends when the laser driver 13 turns off the laser 10, in accordance with the amplified and filtered signal from photocell 14, as described. The time interval during which the laser is energized by driver 13 is proportional to the amplitude of the signal applied to driver 13 via line from amplifier and filter 15, 16. Decoder 19 scales the electrical signal it receives in such a way that the final count in counter 21 at the end of the test interval represents the glucose (or other selected chemical) concentration in standard units. The pulse count accumulated in counter 21 gives an accurate measure of the glucose level of the body specimen under test.

As explained, the present apparatus turns off automatically at the completion of the test interval for a particular body specimen, thus requiring no expert judgment on the part off the person operating the apparatus to conduct the test method of the present invention. The test apparatus is easily handled by a person of no specialized skill, and it does not require frequent re-calibration to yield an accurate measurement of the physiological component that is being tested for.

1. A method of determining the glucose content of a body tissue specimen, which comprises the steps of:
irradiating the specimen with laser light modulated at a predetermined pulse repetition rate to cause said specimen to re-emit light at said pulse repetition rate;
receiving the light re-emitted from the specimen to produce a corresponding electrical signal modulated at said pulse repetition rate;
amplifying and filtering said electrical signal;
controlling said irradiation of the specimen by said amplified and filtered electrical signal;
decoding said amplified and filtered electrical signal to produce a decoded signal;
generating variable frequency pulses in accordance with said decoded signal;
and counting said pulses over an interval of time determined by the specimen irradiation in accordance with said amplified and filtered electrical signal.

2. A method according to claim 1, wherein said amplified and filtered electrical signal controls the duration of laser irradiation of the specimen.

3. A method of measuring the level of a selected physiological component of a body tissue specimen, which comprises the steps of:
directing laser light onto the specimen to cause the specimen to re-emit light;
collecting light coming from the specimen;
and quantifying the collected light from the specimen as a measure of said selected physiological component of the specimen.

4. A method according to claim 3, wherein said selected physiological component is blood glucose.

5. An apparatus for measuring the level of a selected physiological component of a body tissue specimen, which comprises:
means for directing laser light onto the specimen to cause the specimen to re-emit light;
means for collecting light coming from the specimen;
and means for quantifying the collected light from the specimen as a measure of said selected physiological component of in the specimen.

6. Apparatus according to claim 5, wherein said selected physiological component is blood glucose.

7. Apparatus for determining the blood glucose content of a body tissue specimen, which comprises:
a laser for irradiating the specimen with laser light;
a driver for energizing said laser and modulating said laser at a predetermined pulse repetition rate to cause said specimen to re-emit light at said pulse repetition rate;
photoelectric means for receiving the light re-emitted from the specimen to produce a corresponding electrical signal modulated at said pulse repetition rate;
an amplifier connected to said photoelectric means for amplifying and filtering said electrical signal therefrom, said amplifier having an output connected to said driver for determining the duration of energization of the laser by the driver;
a decoder connected to the output of said amplifier for decoding said amplified and filtered electrical signal from the specimen to produce a decoded signal;
a variable pulse generator connected to the output of said decoder for generating variable frequency pulses in accordance with said decoded signal;
and a counter connected to the output of said variable frequency pulse generator for counting said pulses over an interval of time determined by the energization of the laser by said driver.

8. Apparatus according to claim 7, wherein said decoder operates said variable frequency pulse generator so that the final count in said counter at the end of said interval represents the glucose content of said specimen in standard units.
